(19) United States
(12) Patent Application Publication
(10) Pub. No.: US 2012/0238455 A1
(43) Pub. Date: Sep. 20, 2012

(54) METHOD FOR DIAGNOSING DRUG ERUPTION RISK INDUCED BY ANTIEPILEPTIC DRUG BASED ON SINGLE NUCLEOTIDE POLYMORPHISM IN 21.33 REGION OF SHORT ARM OF CHROMOSOME 6

(75) Inventors: Yusuke NAKAMURA, Yokohama-shi (JP); Michiaki KUBO, Yokohama-shi (JP); Taisei MUSHIRODA, Yokohama-shi (JP); Takeshi OZEKI, Yokohama-shi (JP)

(73) Assignee: RIKEN, Wako-shi (JP)

(21) Appl. No.: 13/313,289

(22) Filed: Dec. 7, 2011

(30) Foreign Application Priority Data
Mar. 14, 2011 (JP) 2011-055785

(51) Int. Cl. C40B 20/00; C07H 21/04

(52) U.S. Cl. 506/2; 536/24.31; 536/24.33

(57) ABSTRACT

The genotype of a single nucleotide polymorphism (SNP) existing in the 21.33 region of the short arm of chromosome 6, such as an SNP at the HLA-A locus, is analyzed, and, based on the results, drug eruption risk induced by an antiepileptic drug is diagnosed.

### TECHNICAL FIELD

The present invention relates to a method for diagnosing the drug eruption risk induced by an antiepileptic drug, and a reagent used for the method of diagnosis.

### BACKGROUND ART

Drug eruption is representative of cutaneous adverse drug reactions (cADRs), and characterized as an acute inflammatory reaction of the skin or mucosa caused by drugs. Drug eruption occurs dose-independently and unpredictably, and it is often life-threatening. There are a wide variety of symptoms of drug eruption, including both moderate symptoms and severe symptoms. Examples of the severe symptoms include Stevens-Johnson syndrome (SJS), toxic epidermal necrolysis (TEN), and drug-induced hypersensitivity syndrome (DIHS), which are known as the 3 major severe drug eruptions.

Almost all the drugs have been reported to have the risk of inducing drug eruption, but it is known that, among the drugs, carbamazepine (CBZ), which is an antiepileptic drug, can induce various drug eruptions including SJS, TEN, and DIHS.

Based on the studies so far, it is thought that T cell-type allergic reactions are involved in development of drug eruption, but details of its pathogenic mechanism have not been clarified yet. Further, although reactivation of human herpes virus type 6 (HHV-6) is suggested to be involved in symptoms of DIHS such as fever and hepatitis, its pathogenic mechanism has not been clarified yet.

In terms of CBZ, a research using Taiwanese subjects demonstrated that the human leukocyte antigen (HLA)-B*1502 allele is very strongly associated with SJS and TEN induced by CBZ (Chung W H. et al. Nature. 2004 Apr. 1; 428(6982):486). However, the frequencies of alleles at the HLA loci remarkably vary depending on the race, and, for example, the HLA-B*1502 allele exists in East Asians at a frequency of 8.6% (Chung W H. et al. Nature. 2004 Apr. 1; 428(6982):486), while it exists in Japanese and Caucasians at a frequency of only 0.1% (http://www.allelefrequencies.net). Therefore, in Japanese and Caucasians, the HLA-B*1502 allele cannot be a useful genetic factor for prediction of SJS and TEN induced by CBZ.

Further, there is a literature describing association between the HLA-A*3101 allele and severe drug eruption induced by CBZ in Japanese subjects (Kashiwagi et al., J Dermatol. 2008 October; 35(10):683-5). Although it has been reported that the association was significant (P=0.0004) and the odds ratio is 4.33, the result is uncertain since the sample size in the analysis was small, as the authors themselves recognized. Actually, in a continued report by the same group, the relative risk of the HLA-A*3101 allele was 1.33 in both moderate drug eruption and severe drug eruption, so that the association between the HLA-A*3101 allele and drug eruption induced by CBZ could not be confirmed (Ikeda, et al., Epilepsia. 2010 February; 51(2):297-300).

Further, there is also a report suggesting association between the HLA-A*3101 allele and maculopapular eruption (MPE) induced by CBZ. However, association between the HLA-A*3101 allele and SJS or TEN was not found (Hung, et al., Pharmacogenet Genomics. 2006 April; 16(4):297-306).

Further, it has been reported that, among Caucasian subjects, one case of hypersensitivity syndrome induced by CBZ had the HLA-A*3101 allele (Calligaris, et al., Int Arch Allergy Immunol. 2009; 149(2):173-7). However, this study did not statistically demonstrate the association between the HLA-A*3101 allele and drug eruption induced by CBZ.

Thus, especially in Japanese and Caucasians, no clinical test is known which can be used for predicting the risk of development of drug eruption due to an antiepileptic drug such as CBZ.

### SUMMARY OF THE INVENTION

### Object to be Achieved by the Invention

An object of the present invention is to provide a method for accurately diagnosing the drug eruption risk induced by an antiepileptic drug, and a diagnostic reagent to be used for the method.

### Means for Achieving the Object

As a result of intensive study for solving the above-described problems, the present inventors found that single nucleotide polymorphisms (SNPs) existing in the 21.33 region of the short arm of chromosome 6 were associated with the drug eruption risk induced by carbamazepine (CBZ). The present inventors further found that the genotype at the HLA-A locus existing in the 21.33 region of the short arm of chromosome 6 was associated with the drug eruption risk induced by CBZ. The present inventors then discovered that, by analyzing these polymorphisms, the drug eruption risk induced by an antiepileptic drug such as CBZ can be accurately predicted, thereby completing the present invention.

That is, the present invention provides the followings.
[1] A method for diagnosing drug eruption risk induced by an antiepileptic drug, comprising:

analyzing a single nucleotide polymorphism in the 21.33 region of the short arm of chromosome 6; and

diagnosing drug eruption risk induced by an antiepileptic drug based on a result of said analysis.
[2] The method according to [1], wherein said antiepileptic drug is carbamazepine.
[3] The method according to [1] or [2], wherein said single nucleotide polymorphism is analyzed by analyzing the genotype at the HLA-A locus.
[4] The method according to any one of [1] to [3], wherein whether or not the HLA-A locus has the HLA-A*3101 allele is analyzed.
[5] The method according to any one of [1] to [4], wherein said single nucleotide polymorphism is a polymorphism of (1), (2), or (3) below:

(1) a single nucleotide polymorphism of a nucleotide corresponding to the nucleotide at nucleotide position 61 of a nucleotide sequence selected from SEQ ID NOs: 1 to 12;

(2) a single nucleotide polymorphism showing linkage disequilibrium with said nucleotide;

(3) a single nucleotide polymorphism showing linkage disequilibrium with HLA-A*3101.
[6] A probe for diagnosing drug eruption risk induced by an antiepileptic drug, said probe having a sequence of (1) or (2) below:

(1) a sequence of 10 or more nucleotides in a nucleotide sequence selected from SEQ ID NOs: 1 to 12, said sequence of 10 or more nucleotides comprising the nucleotide at nucleotide position 61, or a complementary sequence thereof;

(2) a sequence of 10 or more nucleotides comprising a single nucleotide polymorphism showing linkage disequilibrium with HLA-A*3101, or a complementary sequence thereof.
[7] A primer for diagnosing drug eruption risk induced by an antiepileptic drug, which primer can amplify a region of (1) or (2) below:

(1) a region in a nucleotide sequence selected from SEQ ID NOs: 1 to 12, said region comprising the nucleotide at nucleotide position 61;

(2) a region comprising a single nucleotide polymorphism showing linkage disequilibrium with HLA-A*3101.

### Effect of the Present Invention

By the present invention, the drug eruption risk induced by an antiepileptic drug can be accurately and simply predicted. Therefore, the present invention is effective for determining whether or not an antiepileptic drug should be administered, and contributes to drug therapy using an antiepileptic drug.

### BRIEF DESCRIPTION OF THE DRAWINGS

FIG. 1 is a diagram showing a linkage disequilibrium (LD) map of the 21.33 region of the short arm of human chromosome 6.

### MODE FOR CARRYING OUT THE INVENTION

### <1> Method of Present Invention

The method of the present invention is a method for diagnosing drug eruption risk induced by an antiepileptic drug, comprising analyzing a single nucleotide polymorphism (SNP) in the 21.33 region of the short arm of chromosome 6 (6p21.33 region), and diagnosing drug eruption risk induced by an antiepileptic drug based on a result of the analysis. In the present invention, the term “drug eruption risk” includes both a risk of occurrence of drug eruption due to administration of an antiepileptic drug and a risk of exacerbation of drug eruption due to administration of an antiepileptic drug. Therefore, the term “diagnosis” in the present invention includes both a diagnosis for predicting whether or not drug eruption occurs due to administration of an antiepileptic drug and diagnosis for predicting whether or not drug eruption is exacerbated due to administration of an antiepileptic drug. In the method of the present invention, a result of analysis of an SNP is correlated with a risk of occurrence of drug eruption due to administration of an antiepileptic drug and/or a risk of exacerbation of drug eruption due to administration of an antiepileptic drug.

Examples of the drug eruption include, but are not limited to, Stevens-Johnson syndrome (SJS), toxic epidermal necrolysis (TEN), drug-induced hypersensitivity syndrome (DIHS), erythema multiforme (EM), maculopapular eruption (MPE), erythema, erythroderma, and fixed drug eruption.

The antiepileptic drug is not particularly restricted and would be, for example, an iminostilbene derivative. The iminostilbene derivative can be, for example, carbamazepine (CBZ).

Specific examples of SNPs existing in the 6p21.33 region include human rs1633021, rs2571375, rs1116221, rs2844796, rs1736971, rs1611133, rs2074475, rs7760172, rs2517673, rs2524005, rs12665039, and rs1362088, and single nucleotide polymorphisms showing linkage disequilibrium with HLA-A*3101. Here, the rs numbers refer to registration numbers for the dbSNP database (http//www.ncbi.nlm.nih.gov/projects/SNP/) by National Center for Biotechnology Information.

rs1633021 means the polymorphism of adenine (A)/guanine (G) of the 20605120th nucleotide in GenBank Accession No. NT_{—}007592.14, and in cases where this nucleotide is G, there is a high drug eruption risk induced by an antiepileptic drug. Further, if the genotype is taken into consideration in the analysis, the drug eruption risk by an antiepileptic drug is higher in the order of rs1633021 of GG>GA>AA.

rs2571375 means the polymorphism of thymine (T)/cytosine (C) of the 20803521st nucleotide in GenBank Accession No. NT_{—}007592.14, and in cases where this nucleotide is C, there is a high drug eruption risk induced by an antiepileptic drug. Further, if the genotype is taken into consideration in the analysis, the drug eruption risk by an antiepileptic drug is higher in the order of rs2571375 of CC>CT>TT.

rs1116221 means the polymorphism of thymine (T)/cytosine (C) of the 20929581st nucleotide in GenBank Accession No. NT_{—}007592.14, and in cases where this nucleotide is T, there is a high drug eruption risk induced by an antiepileptic drug. Further, if the genotype is taken into consideration in the analysis, the drug eruption risk by an antiepileptic drug is higher in the order of rs1116221 of TT>TC>CC.

rs2844796 means the polymorphism of thymine (T)/cytosine (C) of the 20930762nd nucleotide in GenBank Accession No. NT_{—}007592.14, and in cases where this nucleotide is T, there is a high drug eruption risk induced by an antiepileptic drug. Further, if the genotype is taken into consideration in the analysis, the drug eruption risk by an antiepileptic drug is higher in the order of rs2844796 of TT>TC>CC.

rs1736971 means the polymorphism of adenine (A)/cytosine (C) of the 20634573rd nucleotide in GenBank Accession No. NT_{—}007592.14, and in cases where this nucleotide is A, there is a high drug eruption risk induced by an antiepileptic drug. Further, if the genotype is taken into consideration in the analysis, the drug eruption risk by an antiepileptic drug is higher in the order of rs1736971 of AA>AC>CC.

rs1611133 means the polymorphism of thymine (T)/cytosine (C) of the 20667633rd nucleotide in GenBank Accession No. NT_{—}007592.14, and in cases where this nucleotide is T, there is a high drug eruption risk induced by an antiepileptic drug. Further, if the genotype is taken into consideration in the analysis, the drug eruption risk by an antiepileptic drug is higher in the order of rs1611133 of TT>TC>CC.

rs2074475 means the polymorphism of adenine (A)/guanine (G) of the 20996141st nucleotide in GenBank Accession No. NT_{—}007592.14, and in cases where this nucleotide is G, there is a high drug eruption risk induced by an antiepileptic drug. Further, if the genotype is taken into consideration in the analysis, the drug eruption risk by an antiepileptic drug is higher in the order of rs2074475 of GG>GA>AA.

rs7760172 means the polymorphism of thymine (T)/cytosine (C) of the 20688325th nucleotide in GenBank Accession No. NT_{—}007592.14, and in cases where this nucleotide is C, there is a high drug eruption risk induced by an antiepileptic drug. Further, if the genotype is taken into consideration in the analysis, the drug eruption risk by an antiepileptic drug is higher in the order of rs7760172 of CC>CT>TT.

rs2517673 means the polymorphism of thymine (T)/cytosine (C) of the 20795493rd nucleotide in GenBank Accession No. NT_{—}007592.14, and in cases where this nucleotide is T, there is a high drug eruption risk induced by an antiepileptic drug. Further, if the genotype is taken into consideration in the analysis, the drug eruption risk by an antiepileptic drug is higher in the order of rs2517673 of TT>TC>CC.

rs2524005 means the polymorphism of thymine (T)/cytosine (C) of the 20757928th nucleotide in GenBank Accession No. NT_{—}007592.14, and in cases where this nucleotide is T, there is a high drug eruption risk induced by an antiepileptic drug. Further, if the genotype is taken into consideration in the analysis, the drug eruption risk by an antiepileptic drug is higher in the order of rs2524005 of TT>TC>CC.

rs12665039 means the polymorphism of thymine (T)/cytosine (C) of the 20783030th nucleotide in GenBank Accession No. NT_{—}007592.14, and in cases where this nucleotide is C, there is a high drug eruption risk induced by an antiepileptic drug. Further, if the genotype is taken into consideration in the analysis, the drug eruption risk by an antiepileptic drug is higher in the order of rs12665039 of CC>CT>TT.

rs1362088 means the polymorphism of adenine (A)/guanine (G) of the 21068087th nucleotide in GenBank Accession No. NT_{—}007592.14, and in cases where this nucleotide is G, there is a high drug eruption risk induced by an antiepileptic drug. Further, if the genotype is taken into consideration in the analysis, the drug eruption risk by an antiepileptic drug is higher in the order of rs1362088 of GG>GA>AA.

For rs1633021, rs2571375, rs1116221, rs2844796, rs1736971, rs1611133, rs2074475, rs7760172, rs2517673, rs2524005, rs12665039 and rs1362088, nucleotide sequences having a total length of 121 by are shown in SEQ ID NOs: 1 to 12, respectively, each of which comprises the SNP nucleotide and the sequences of the upstream and downstream regions thereof each of which has a length of 60 bp. The 61st nucleotide has the polymorphism.

In the present invention, a nucleotide corresponding to the above-described nucleotide is analyzed. The term “nucleotide corresponding to the above-described nucleotide” means a corresponding nucleotide in the above-described region. That is, “analysis of a nucleotide corresponding to the above-described nucleotide” includes analysis of a corresponding nucleotide in the above-described region even in cases where the above-described sequence is somewhat changed at positions other than that of the SNP due to difference in the race and/or the like.

The nucleotide to be analyzed in the present invention is not restricted to the above-described nucleotide, and a polymorphism of a nucleotide showing linkage disequilibrium with the above-described nucleotide may also be analyzed. Here, the term “nucleotide showing linkage disequilibrium with the above-described nucleotide” means a nucleotide satisfying the relationship of, for example, r²>0.5, r²>0.8, or r²>0.9 with the above-described nucleotide. As for any of nucleotides showing linkage disequilibrium with the above-described nucleotide(s), the drug eruption risk induced by an antiepileptic drug is higher in the order of: the homozygote of risk alleles>the heterozygote of a risk allele and a non-risk allele>the homozygote of non-risk alleles.

Nucleotides showing linkage disequilibrium with the above-described nucleotide(s) can be identified using HapMap database (http://www.hapmap.org/index.html.ja) or the like. Further, these can also be identified by analyzing sequences of DNAs collected from a plurality of subjects (usually about 20 to 40 individuals) using a sequencer and then searching SNPs showing linkage disequilibrium.

By analyzing the type of the nucleotide of the above-described SNP and relating an obtained result to the drug eruption risk induced by an antiepileptic drug based on the above standards, the drug eruption risk induced by the antiepileptic drug can be diagnosed. One of the above-described SNPs may be analyzed solely, or a plurality of SNPs comprising at least one of the above-described SNPs may be analyzed at the same time (i.e. haplotype analysis). For example, a plurality of the above-described SNPs may be analyzed at the same time, or at least one of the above-described SNPs and at least one of the other SNPs associated with the drug eruption risk induced by the antiepileptic drug may be analyzed in combination. By analyzing a plurality of SNPs associated with the drug eruption risk induced by the antiepileptic drug at the same time, accuracy of the diagnosis of the drug eruption risk induced by the antiepileptic drug can be increased. For any SNP, either strand of the double-stranded DNA may be analyzed.

The 21.33 region of the short arm of human chromosome 6 contains the HLA-A gene. Since the genotype at the HLA-A locus is determined by the combination of genotypes of SNPs in the HLA-A locus, SNPs contained in the 21.33 region of the short arm of human chromosome 6, more particularly, SNPs in the HLA-A locus, can be analyzed by analyzing the genotype at the HLA-A locus. That is, an aspect of the method of the present invention is a method for diagnosing drug eruption risk induced by an antiepileptic drug, comprising analyzing the genotype at the HLA-A locus, and diagnosing drug eruption risk induced by an antiepileptic drug based on a result of the analysis.

The HLA-A gene encodes the heavy chain of the HLA class I molecule. Particular examples of the HLA-A locus include the region of 29910309 to 29913661 in GenBank Accession No. NC_{—}000006.11. In cases where a subject has HLA-A*3101 allele, there is a high drug eruption risk induced by an antiepileptic drug.

By analyzing the genotype at the HLA-A locus and relating an obtained result to the drug eruption risk induced by an antiepileptic drug, the drug eruption risk induced by the antiepileptic drug can be diagnosed. The HLA-A gene may be analyzed for either strand of the double-stranded DNA. Further, at least one of SNPs associated with the drug eruption risk induced by the antiepileptic drug and the genotype at the HLA-A locus may be analyzed at the same time.

The sample to be used for the analysis of an SNP(s) or analysis of HLA-A alleles (hereinafter collectively referred to as analysis of polymorphisms) is not restricted as long as the sample contains the chromosomal DNA, and examples of the sample include body fluid samples such as blood and urine; cells such as those of oral mucosa; and body hairs such as hairs. These samples may be directly used for the analysis of polymorphisms, or for example, the chromosomal DNA can be isolated from these samples according to a conventional method and this isolated DNA can be used for the analysis.

Analysis of the polymorphisms can be carried out by a conventional method for analysis of genetic polymorphisms. Examples of the method include, but are not limited to, sequencing analysis, PCR, hybridization, and the invader method. In cases where HLA-A alleles are to be analyzed, either the entire or a partial sequence of the HLA-A gene may be analyzed as long as HLA-A*3101 can be distinguished from the other HLA-A alleles.

The sequencing analysis may be carried out by a conventional method. More particularly, by carrying out sequencing reaction using a primer to be located at a position which is several ten nucleotides upstream of a nucleotide showing polymorphism, the type of the nucleotide at the corresponding position can be determined based on the result of the analysis. The term “nucleotide showing polymorphism” means'a polymorphic nucleotide at the HLA-A locus which can be used for determining the above-described SNP nucleotide or the genotype at the HLA-A locus. Particular examples of the polymorphic nucleotide which can be used for determining the genotype at the HLA-A locus include polymorphic nucleotides which can be used for distinguishing HLA-A*3101 from the other HLA-A alleles. More particular examples of such a polymorphic nucleotide include single nucleotide polymorphisms showing linkage disequilibrium with HLA-A*3101. The single nucleotide polymorphisms showing linkage disequilibrium with HLA-A*3101 mean nucleotides satisfying the relationship of, for example, r²>0.5, r²>0.8, or r²>0.9 with HLA-A*3101. The position where “a nucleotide showing polymorphism” exists is also referred to as “a polymorphic site”. For example, before the sequencing reaction, a DNA fragment containing the polymorphic site can be preliminarily amplified by PCR or the like.

Analysis of polymorphisms can be carried out by investigating amplification by PCR. For example, primers having a sequence corresponding to a region containing a polymorphic nucleotide, whose 3′-ends correspond to the respective polymorphisms, are prepared. Using each primer, PCR is carried out, and based on the presence or absence of an amplified product, the type of the SNP or HLA-A allele can be determined. Further, whether or not the amplification occurs may also be investigated by the LAMP method (JP 3313358 B), NASBA method (Nucleic Acid Sequence-Based Amplification; JP 2843586 B), ICAN method (JP 2002-233379 A), or the like. The single chain amplification method may also be used.

Further, by amplifying a DNA fragment containing a polymorphic site and investigating the difference in the mobility of the amplified product in electrophoresis, the type of the polymorphism can be determined. Examples of such a method include the PCR-SSCP (single-strand conformation polymorphism) method (Genomics. 1992 Jan. 1; 12(1): 139-146). More particularly, DNA containing a polymorphic site of interest is amplified, and the amplified DNA is dissociated into single-stranded DNAs. Thereafter, the dissociated single-stranded DNAs are separated on a non-denaturing gel, and, based on the difference in the mobility of the separated single-stranded DNAs on the gel, the type of the SNP or HLA-A allele can be determined.

Further, in cases where the nucleotide showing polymorphism is contained in a restriction enzyme recognition sequence, analysis of polymorphisms can be carried out by investigating whether or not digestion by the restriction enzyme occurs (RFLP method). In this case, firstly, a DNA sample is digested with a restriction enzyme. Subsequently, DNA fragment(s) are separated, and the type of the SNP or HLA-A allele can be determined based on the sizes of the detected DNA fragment(s).

Further, analysis of polymorphisms can be carried out by investigating whether or not hybridization occurs. That is, probes corresponding to the respective polymorphisms are prepared, and with which probe the DNA hybridizes is investigated, thereby determining the type of the SNP or HLA-A allele.

By determining the type of the SNP or HLA-A allele as described above, data for diagnosing the drug eruption risk induced by an antiepileptic drug can be obtained.

### <2> Diagnostic Reagent of Present Invention

The present invention also provides diagnostic reagents such as primers and probes for diagnosing the drug eruption risk induced by an antiepileptic drug. Examples of such probes include a probe comprising the above-described polymorphic site, which allows determination of the type of the nucleotide at the polymorphic site based on whether or not hybridization occurs. Particular examples of the probes include a probe having a sequence of 10 or more nucleotides in a nucleotide sequence selected from SEQ ID NOs: 1 to 12, which sequence of 10 or more nucleotides comprises the nucleotide at nucleotide position 61, or the complementary sequence thereof; and a probe having a sequence of 10 or more nucleotides comprising a polymorphic nucleotide at the HLA-A locus. The length of the probe is, for example, 15 to 35 nucleotides, or 20 to 35 nucleotides. The polymorphic nucleotide at the HLA-A locus can be, for example, a single nucleotide polymorphism showing linkage disequilibrium with HLA-A*3101.

Examples of the primers include a primer which can be used for PCR for amplifying the above-described polymorphic site and a primer which can be used for sequencing analysis of the above-described polymorphic site. More particular examples of the primers include a primer which allows amplification or sequencing of a region in a nucleotide sequence selected from SEQ ID NOs: 1 to 12, which region comprises the nucleotide at nucleotide position 61 and a primer which allows amplification or sequencing of a region comprising a polymorphic nucleotide at the HLA-A locus. The length of the primer is, for example, 10 to 50 nucleotides, 15 to 35 nucleotides, or 20 to 35 nucleotides. The polymorphic nucleotide at the HLA-A locus can be, for example, a single nucleotide polymorphism showing linkage disequilibrium with HLA-A*3101.

Examples of the primer for sequencing the above-described polymorphic site include a primer having a sequence of the 5′ region of the above-described polymorphic nucleotide, or for example, a sequence of 30 to 100 nucleotides upstream region of the above-described polymorphic nucleotide, and a primer having a complementary sequence of the 3′ region of the above-described polymorphic nucleotide, or for example, a complementary sequence of 30 to 100 nucleotides downstream region of the above-described polymorphic nucleotide. Examples of the primer to be used for determining a polymorphism based on whether or not amplification by PCR occurs include a primer having a sequence comprising the above-described polymorphic nucleotide, wherein the nucleotide is contained in the 3′-side of the primer, and a primer having a complementary sequence of a sequence comprising the above-described polymorphic nucleotide, wherein the nucleotide is contained in the 3′-side of the primer.

The diagnostic reagents of the present invention may comprise, in addition to the primer(s) and/or the probe(s), a polymerase and/or a buffer for PCR, reagents for hybridization, and/or the like.

### EXAMPLES

The present invention will now be described in more detail. However, the present invention is not restricted to these Examples.

(1) Identification of SNPs Associated with Drug Eruption Risk induced by Carbamazepine (CBZ)

In order to identify genetic polymorphisms which determine drug eruption risk induced by CBZ, a genome-wide association study (GWAS) was carried out using Japanese subjects, and, based on the obtained results, analysis of the genotype at the HLA-A locus and a replication study were carried out. GWAS is a genetic statistical method for searching genetic polymorphisms involved in phenotypes such as diseases. Genetic polymorphisms associated with a disease can be discovered by, for example, using SNPs at several hundred thousand to one million sites covering the whole human genome to statistically test whether or not there are differences in the frequencies of the polymorphisms between patients suffering from the disease (cases) and subjects who are not suffering from the disease (controls).

### <Subjects>

For GWAS and the genotype analysis of the HLA-A locus, 62 subjects with drug eruption induced by CBZ (drug eruption subjects (cases)) were used. Among the 62 cases, 33 subjects with non-DIHS cutaneous reactions induced by CBZ were selected from patients registered in BioBank Japan (BBJ) (Nakamura, Y. The BioBank Japan Project. Clin Adv Hematol Oncol 5, 696-7 (2007)) of Institute of Medical Science, University of Tokyo. Among the 33 subjects, 4 subjects were suffering from Stevens-Johnson syndrome (SJS) or toxic epidermal necrolysis (TEN), 16 subjects were suffering from erythema multiforme (EM), 4 subjects were suffering from maculopapular eruption (MPE), 2 subjects were suffering from erythema, 1 subject was suffering from erythroderma, 1 subject was suffering from fixed drug eruption, and 5 subjects were suffering from unclassified drug eruptions. SJS is defined by detachment of less than 10% of the skin of the body surface, and TEN is defined by detachment of more than 10% of the skin of the body surface (in either case, staphylococcal scalded skin syndrome is excluded). Further, 29 subjects with typical DIHS induced by CBZ among the 62 cases were obtained from Yokohama City University Hospital, Showa University Hospital, Kyorin University Hospital, and Ehime University Hospital.

For GWAS and the genotype analysis of the HLA-A locus, the following 2 groups of control subjects were used.

As the first group of control subjects, 898 volunteers recruited from Osaka-Midosuji Rotary Club and its related Rotary Clubs were used as the population of general Japanese individuals. The 898 volunteers had no history of epilepsy, cranial neuropathy, cancer, and therapy by CBZ.

As the second group of control subjects, 376 subjects who showed no drug eruptions by administration of CBZ (CBZ-tolerant controls) were obtained from BBJ.

In the replication study, 16 drug eruption subjects and 44 CBZ-tolerant controls obtained from Yokohama City University Hospital, Showa University Hospital, Kyorin University Hospital, and Ehime University Hospital were used.

The present research was approved by Human Genome Ethics Review Committee of Institute of Medical Science, University of Tokyo, and Research Ethics Committee of RIKEN Yokohama Institute, and informed consent was obtained from all the subjects.

### <Statistical Analysis>

The association between each SNP or each HLA allele and drug eruption induced by CBZ (CBZ-induced drug eruption) was evaluated by Fisher's exact test. For GWAS, the association study was carried out using the allele frequency model, dominant-inheritance model and recessive-inheritance model. The association between each SNP and CBZ-induced drug eruption was evaluated based on the lowest P-value calculated based on these 3 models.

### <GWAS>

The genotypes in the 55 drug eruption subjects and 898 general population subjects were analyzed using HumanHap550v3 Genotyping BeadChip (Illumina, Inc.). For the association study, 1 drug eruption subject and 16 general population subjects who were judged as outliers by principal component analysis (PCA) were excluded, and further, 1 drug eruption subject was excluded for quality control. Data from the remaining 53 drug eruption subjects and 882 general population subjects were employed. The association study was carried out for 444823 autosomal SNPs, which have passed through quality control, among the 554496 SNPs of which the genotypes were analyzed.

As a result of GWAS, 12 SNPs significantly associated with CBZ-induced drug eruption were identified, which SNPs satisfied P<1.12×10⁻⁷ (=0.05/444823) after Bonferroni correction in multiple testing (Table 1). Among these SNPs, rs1633021 showed the lowest P-value, and this SNP was therefore revealed to be most strongly associated with CBZ-induced drug eruption (P=1.18×10⁻¹³). To verify this result, rs1633021 was subjected to multiplex-PCR invader assay (Third Wave Technologies) (Ohnishi, Y. et al. J Hum Genet 46, 471-7 (2001)), and 100% matching with the result of GWAS was confirmed.

TABLE 1
SNPs associated with CBZ-induced drug eruption

| | | | Case | | | | General population | | | | P-value | | |
|---|---|---|---|---|---|---|---|---|---|---|---|---|---|
| SNP | Chr | Allele (1/2) | 11 | 12 | 22 | MAF | 11 | 12 | 22 | MAF | 11 vs 12 + 22 | 11 + 12 vs 22 | 1 vs 2 |
| rs1633021 | 6 | A/G | 19 | 32 | 2 | 0.340 | 736 | 142 | 3 | 0.084 | 1.18 × 10⁻¹³ | 2.83 × 10⁻² | 1.58 × 10⁻¹² |
| rs2571375 | 6 | T/C | 23 | 26 | 4 | 0.321 | 736 | 143 | 3 | 0.084 | 2.44 × 10⁻¹⁰ | 2.85 × 10⁻⁴ | 3.82 × 10⁻¹¹ |
| rs1116221 | 6 | T/C | 4 | 26 | 23 | 0.321 | 4 | 149 | 729 | 0.089 | 5.46 × 10⁻⁴ | 7.12 × 10⁻¹⁰ | 1.35 × 10⁻¹⁰ |
| rs2844796 | 6 | T/C | 4 | 26 | 23 | 0.321 | 4 | 150 | 728 | 0.090 | 5.46 × 10⁻⁴ | 8.26 × 10⁻¹⁰ | 1.58 × 10⁻¹⁰ |
| rs1736971 | 6 | A/C | 6 | 35 | 12 | 0.443 | 27 | 282 | 573 | 0.190 | 8.43 × 10⁻³ | 1.49 × 10⁻⁹ | 1.46 × 10⁻⁸ |
| rs1611133 | 6 | T/C | 6 | 35 | 12 | 0.443 | 27 | 285 | 570 | 0.192 | 8.43 × 10⁻³ | 1.90 × 10⁻⁹ | 1.63 × 10⁻⁸ |
| rs2074475 | 6 | A/G | 28 | 21 | 4 | 0.274 | 750 | 129 | 3 | 0.077 | 9.99 × 10⁻⁸ | 2.85 × 10⁻⁴ | 5.60 × 10⁻⁹ |
| rs7760172 | 6 | T/C | 16 | 31 | 6 | 0.406 | 622 | 236 | 24 | 0.161 | 6.07 × 10⁻⁹ | 5.16 × 10⁻³ | 7.05 × 10⁻⁹ |
| rs2517673 | 6 | T/C | 6 | 31 | 16 | 0.406 | 24 | 237 | 621 | 0.162 | 5.16 × 10⁻³ | 6.61 × 10⁻⁹ | 7.67 × 10⁻⁹ |
| rs2524005 | 6 | T/C | 6 | 31 | 16 | 0.406 | 24 | 244 | 614 | 0.166 | 5.16 × 10⁻³ | 1.28 × 10⁻⁸ | 1.43 × 10⁻⁸ |
| rs12665039 | 6 | T/C | 17 | 30 | 6 | 0.396 | 621 | 236 | 25 | 0.162 | 4.96 × 10⁻⁸ | 6.12 × 10⁻³ | 4.19 × 10⁻⁸ |
| rs1362088 | 6 | A/G | 29 | 20 | 4 | 0.264 | 742 | 134 | 6 | 0.083 | 1.19 × 10⁻⁶ | 1.50 × 10⁻³ | 9.86 × 10⁻⁸ |

Results of association analysis with 53 drug eruption subjects and 882 general population subjects
MAF: Minor Allele Frequency
Chr: Chromosome

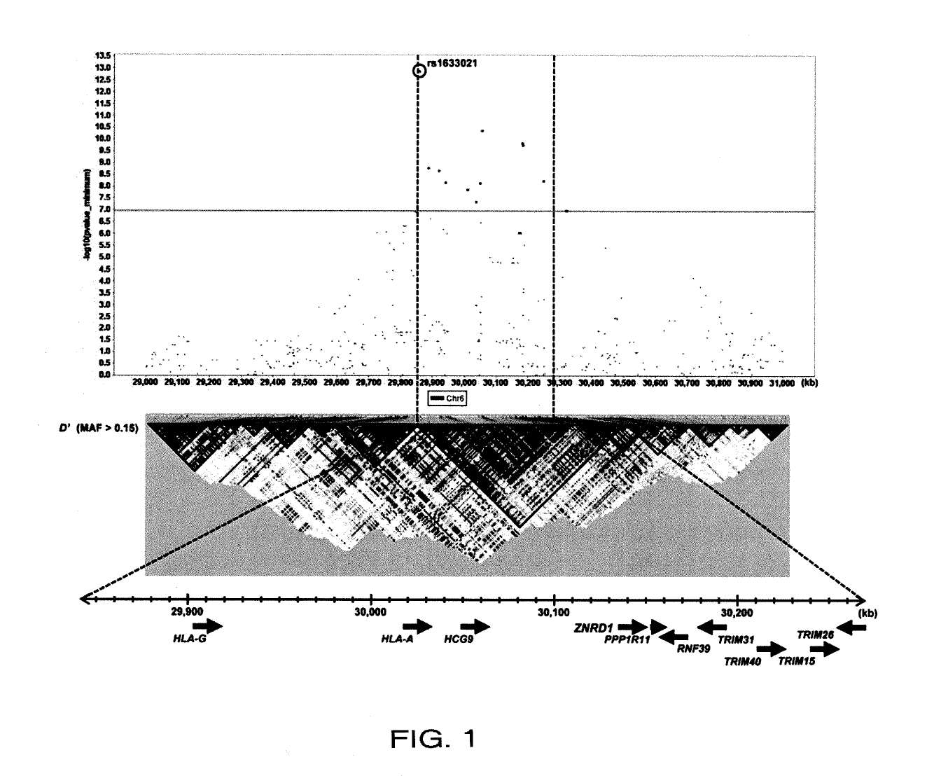

All of these 12 SNPs were located in a region of about 463 kb in the 21.33 region of the short arm of chromosome 6 (6p21.33 region). Thus, using 882 general population subjects, a linkage disequilibrium (LD) map of this region was prepared (FIG. 1). As a result, it was revealed that, among the 12 SNPs, 11 SNPs are located in the single LD block of 29.84 to 30.27 Mb. Further, the remaining 1 SNP (rs1362088) was located near this LD block. This region corresponds to the MHC I region containing the HLA-A locus.

### <Genotype Analysis of HLA-A Locus>

Since SNPs located near the HLA-A locus were found to be associated with CBZ-induced drug eruption, genotype analysis of the HLA-A locus was subsequently carried out. As drug eruption subjects, 61 subjects were used, wherein 7 subjects were added to the above-described 54 subjects selected by excluding 1 drug eruption subject who was judged as an outlier by principal component analysis (PCA). As control subjects, 376 CBZ-tolerant subjects were used.

The results are shown in Table 2. In Table 2, “*” indicates significant association with CBZ-induced drug eruption suggested by satisfaction of P<2.63×10⁻³ (=0.05/19) after Bonferroni correction in multiple testing.

TABLE 2
Frequencies of HLA-A alleles in drug eruption subjects and CBZ-tolerant
subjects

| | Number of carriers | | |
|---|---|---|---|
| HLA allele | Case (%) | CBZ-tolerant control (%) | P-value |
| A*0101 | 0 (0.0) | 6 (1.6) | 1.00 |
| A*0201 | 5 (8.2) | 93 (24.7) | 2.74 × 10⁻³ |
| A*0206 | 1 (1.6) | 68 (18.1) | 2.46 × 10⁻⁴ * |
| A*0207 | 3 (4.9) | 23 (6.1) | 0.10 |
| A*0210 | 2 (3.3) | 2 (0.5) | 0.03 |
| A*0301 | 0 (0.0) | 2 (0.5) | 1.00 |
| A*1101 | 7 (11.5) | 68 (18.1) | 0.27 |
| A*1110 | 0 (0.0) | 1 (0.3) | 1.00 |
| A*2402 | 37 (60.7) | 211 (56.1) | 0.58 |
| A*2405 | 0 (0.0) | 1 (0.3) | 1.00 |
| A*2420 | 0 (0.0) | 4 (1.1) | 1.00 |
| A*2601 | 2 (3.3) | 65 (17.3) | 3.36 × 10⁻³ |
| A*2602 | 2 (3.3) | 15 (4.0) | 1.00 |
| A*2603 | 11 (18.0) | 22 (5.9) | 2.61 × 10⁻³ |
| A*2605 | 1 (1.6) | 1 (0.3) | 0.26 |
| A*2901 | 0 (0.0) | 1 (0.3) | 1.00 |
| A*3001 | 0 (0.0) | 2 (0.5) | 1.00 |
| A*3101 | 37 (60.7) | 47 (12.5) | 3.64 × 10⁻¹⁵ * |
| A*3303 | 5 (8.2) | 59 (15.7) | 0.17 |

The frequency of HLA-A*3101 allele was significantly higher in the drug eruption subjects compared to the CBZ-tolerant subjects (P=3.64×10⁻¹⁵), and the CBZ drug eruption subjects showed an allele frequency of 60.7%, while the CBZ-tolerant subjects showed an allele frequency of only 12.5%. This indicates that the HLA-A*3101 allele has a sensitivity of 60.7% and a specificity of 87.5% as a predictive factor for CBZ-induced drug eruption in Japanese. Here, if the incidence rate of CBZ-induced drug eruption is 2.9%, the positive predictive value is calculated to be 12.7% and the negative predictive value is calculated to be 98.7%. That is, by excluding patients who were judged to be HLA-A*3101-positive from CBZ therapy, the frequency of CBZ-induced drug eruption can be reduced from 2.9% to 1.1%.

Examples of drugs which can be used as alternatives to CBZ for epilepsy and trigeminal neuralgia include drugs showing low incidence rates of drug eruption, such as phenytoin and valproic acid. Although these alternative drugs are inferior to CBZ in terms of the therapeutic effect, these alternative drugs may be more preferable to patients than CBZ in view of prevention of development of drug eruption, which is often life-threatening. Therefore, prediction of the drug eruption risk induced by CBZ by typing of HLA-A*3101 is very useful for determination of the individual therapeutic strategies for diseases such as epilepsy and trigeminal neuralgia.

Further, the frequency of the HLA-A*0206 allele was also significantly higher in the drug eruption subjects than in the CBZ-tolerant subjects (P=2.46×10⁻⁴).

### <Replication Study >

In order to verify the significant association between the HLA-A*3101 allele or the HLA-A*0206 allele and CBZ-induced drug eruption, a replication study was carried out using independent populations. As subjects, 16 drug eruption subjects and 44 CBZ-tolerant subjects were used.

The results are shown in Table 3. In Table 3, “*” indicates significant association with CBZ-induced drug eruption suggested by satisfaction of P<2.50×10⁻² (=0.05/2) after Bonferroni correction in multiple testing.

TABLE 3
Association of HLA-A*3101 and the HLA-A*0206 alleles with CBZ-induced drug eruption

| | HLA-A*0206 | | | | HLA-A*3101 | | | |
|---|---|---|---|---|---|---|---|---|
| | | CBZ-tolerant | | Odds ratio | | CBZ-tolerant | | Odds ratio |
| Population | Case (%) | controls (%) | P-value | (95% CI) | Case (%) | controls (%) | P-value | (95% CI) |
| First study | 1/61 (1.6) | 68/376 (18.1) | 2.46 × 10⁻⁴* | 0.1 | 37/61 (60.7) | 47/376 (12.5) | 3.64 × 10⁻¹⁵* | 10.8 |
| | | | | (0.0-0.6) | | | | (5.9-19.6) |
| Replication | 2/16 (12.5) | 8/44 (18.2) | 0.72 | 0.6 | 8/16 (50.0) | 7/44 (15.9) | 1.53 × 10⁻²* | 5.3 |
| study | | | | (0.1-3.4) | | | | (1.5-24.5) |
| Combined | 3/77 (3.9) | 76/420 (18.1) | 1.02 × 10⁻³* | 0.2 | 45/77 (58.4) | 54/420 (12.9) | 1.09 × 10⁻¹⁶* | 9.5 |
| analysis | | | | (0.1-0.6) | | | | (5.6-16.3) |

CI: Confidence Interval

The HLA-A*3101 allele showed P=1.53×10⁻² (P=1.09×10⁻¹⁶ based on the combined analysis of the primary study and the replication study), and therefore the association with CBZ-induced drug eruption was reproduced. On the other hand, the association between the HLA-A*0206 allele and CBZ-induced drug eruption was not reproduced.

### <Analysis of Associations Between HLA-A*3101 Allele and Various Types of Drug Eruption>

Further, the subjects used for the above studies were combined to analyze associations between the HLA-A*3101 allele and various types of drug eruption. As a result, the HLA-A*3101 allele showed a significant association with each of DIHS (P=2.06×10⁻⁹), SJS/TEN (P=2.35×10⁻⁴), and the other types of drug eruption (P=4.74×10⁻⁸) (Table 4).

TABLE 4
Associations of the HLA-A*3101 allele with various types of drug eruption

| | Number of patients | | | | |
|---|---|---|---|---|---|
| | Positive for | Negative for | | | Odds ratio |
| Subgroup | HLA-A*3101 | HLA-A*3101 | Total | P-value | (95% CI) |
| All CBZ-induced | 45 | 32 | 77 | 1.09 × 10⁻¹⁶* | 9.5 (5.6-16.3) |
| cADRs | | | | | |
| DIHS | 21 | 15 | 36 | 2.06 × 10⁻⁹* | 9.5 (4.6-19.5) |
| SJS/TEN | 5 | 1 | 6 | 2.35 × 10⁻⁴* | 33.9 (3.9-295.6) |
| Others | 19 | 16 | 35 | 4.74 × 10⁻⁸* | 8.0 (3.9-16.6) |
| CBZ-tolerant | 54 | 366 | 420 | — | — |
| controls | | | | | |

“*” indicates significant association with each CBZ-induced drug eruption suggested by satisfaction of the threshold of significance after Bonferroni correction in multiple testing.
cADRs: cutaneous Adverse Drug Reactions
SIS/TEN: Stevens-Johnson Syndrome/Toxic Epidermal Necrolysis
DIHS: Drug-induced Hypersensitivity Syndrome
CI: Confidence Interval

The HLA-A gene encodes the heavy chain of HLA class I, and HLA class I molecules play a central role in the immune system by presenting antigen peptides. Therefore, it is thought that SNPs associated with CBZ-induced drug eruption reflect variations in antigen-binding affinities of HLA-A, and therefore influence the immune response upon development of drug eruption.

Although, in GWAS in the present Examples, the HLA-B locus was not found to have association with CBZ-induced drug eruption, the HLA-B*1502 allele is known to be strongly associated with SJS/TEN induced by CBZ in Taiwanese. Thus, 61 drug eruption subjects and 376 CBZ-tolerant subjects were used to analyze the genotype of the HLA-B locus, but the HLA-B*1502 allele was not found in any of the drug eruption subjects, and no HLA-B allele was found to have association with CBZ-induced drug eruption.

### INDUSTRIAL APPLICABILITY

As described above, 12 SNPs associated with CBZ-induced drug eruption were discovered, and it was revealed that the HLA-A*3101 allele was associated with CBZ-induced drug eruption. Therefore, the HLA-A*3101 allele and these SNPs are useful for diagnosis of the drug eruption risk induced by an antiepileptic drug such as CBZ. Thus, the present invention is effective for determination of whether or not an antiepileptic drug should be administered, and contributes to drug therapy using an antiepileptic drug.

1. A method for diagnosing drug eruption risk induced by an antiepileptic drug, comprising:
analyzing a single nucleotide polymorphism in the 21.33 region of the short arm of chromosome 6; and
diagnosing drug eruption risk induced by an antiepileptic drug based on a result of said analysis.

2. The method according to claim 1, wherein said antiepileptic drug is carbamazepine.

3. The method according to claim 1, wherein said single nucleotide polymorphism is analyzed by analyzing the genotype at the HLA-A locus.

4. The method according to claim 3, wherein whether or not the HLA-A locus has the HLA-A*3101 allele is analyzed.

5. The method according to claim 1, wherein said single nucleotide polymorphism is a polymorphism of (1), (2), or (3) below:
(1) a single nucleotide polymorphism of a nucleotide corresponding to the nucleotide at nucleotide position 61 of a nucleotide sequence selected from SEQ ID NOs: 1 to 12;
(2) a single nucleotide polymorphism showing linkage disequilibrium with said nucleotide;
(3) a single nucleotide polymorphism showing linkage disequilibrium with HLA-A*3101.

6. A probe for diagnosing drug eruption risk induced by an antiepileptic drug, said probe having a sequence of (1) or (2) below:
(1) a sequence of 10 or more nucleotides in a nucleotide sequence selected from SEQ ID NOs: 1 to 12, said sequence of 10 or more nucleotides comprising the nucleotide at nucleotide position 61, or a complementary sequence thereof;
(2) a sequence of 10 or more nucleotides comprising a single nucleotide polymorphism showing linkage disequilibrium with HLA-A*3101, or a complementary sequence thereof.

7. A primer for diagnosing drug eruption risk induced by an antiepileptic drug, which primer can amplify a region of (1) or (2) below:
(1) a region in a nucleotide sequence selected from SEQ ID NOs: 1 to 12, said region comprising the nucleotide at nucleotide position 61;
(2) a region comprising a single nucleotide polymorphism showing linkage disequilibrium with HLA-A*3101.


SEQUENCE LISTING (12 sequences)

SEQ ID NO: 1 - DNA, 121 bp, Homo sapiens
attttgaatcctccttcaagcatatatagaggcggcagaattcagtttctgacagtcgttragactgagtttcctgttccctgctagctgtcaaggtagagaggagggagggctgtgctca

SEQ ID NO: 2 - DNA, 121 bp, Homo sapiens
aaaataaaattagaaataataaaactacatctttcagatattattctcaccttcacctttyctctccccttttctccctctcccttcctttcctcgaaatgtccccccatccttccctcct

SEQ ID NO: 3 - DNA, 121 bp, Homo sapiens
tccgaggagctccatgcggcactgagtgagtggctgacagcgatccgggcttggttttgtraggttccttcaagctaagccagctcagagaacacggggagcggtggtgctccacggactt

SEQ ID NO: 4 - DNA, 121 bp, Homo sapiens
gagacccactgtaataacagcctgtgctaaagtcattagatatggttctgccacctaagayaataacagtagaagtaccatttcttaaaggaccactatgtgcgcatctaatatttcaaaa

SEQ ID NO: 5 - DNA, 121 bp, Homo sapiens
tagatctcttggtggagaattgtcatcttaacaatattattgagctttccaaccgcatctmtccatgaatttagacctttaatttctctcagtaatgttttgtagttttcaacaaactctg

SEQ ID NO: 6 - DNA, 121 bp, Homo sapiens
tacacataaacacaaaggataccaccgtaaccatttaaagtgcacaatgcagtgacaattygtatgttcacaatgttatgtaaccatcatcactctctagttccagagtgtttttatcacc

SEQ ID NO: 7 - DNA, 121 bp, Homo sapiens
agccttaaactacccttgtgtatgctgctatttgtaaaggaaaagaggctagaagggaagraaaaaaaaggatcaacgaaaatatatattgcaatgaaaaaaactcctgattctggtccca

SEQ ID NO: 8 - DNA, 121 bp, Homo sapiens
aaatgacctaatcagcaattgctggaaaccaaagtaacagtatacaattctatgtgttaayatcatataccaagagagaaaatgtatagatatcagtagtagccatttaagcactgtaata

SEQ ID NO: 9 - DNA, 121 bp, Homo sapiens
gctgttaagaccccagccataatcacgagagatgtaagaattgaaggtaccattcctttcyatttttcaaaccaaccttctaaccagttagtaaaaaggtcatttactcctgtgttttctg

SEQ ID NO: 10 - DNA, 121 bp, Homo sapiens
ttgctgtctgcccatcttttctgaccctgggacaccaccctttattaagctttcccataaytccttgaaggtcaagcccccttgactgatactctgggttatcacagtaagtgtgccctct

SEQ ID NO: 11 - DNA, 121 bp, Homo sapiens
acgctaaaacatccctctccctcttgtgaggaggaagagtcctcccgggttttcagatccyatactagagagtgactgagggcctgccctgcactctgggacagttaaaggatgaagtctc

SEQ ID NO: 12 - DNA, 121 bp, Homo sapiens
tgctccaacaggtactgttccctttctctcaaaaaccgatggccctattcaaacactgatrcaatgtcttgcctgtggttctgaaatgttgtctccagtaaatgcagaaagagaaaagtga